(19) United States
(12) Patent Application Publication
(10) Pub. No.: US 2005/0234442 A1
(43) Pub. Date: Oct. 20, 2005

(54) Electrosurgical cutting and cauterizing device

(76) Inventor: Michael Spears, Canton, MI (US)

Correspondence Address: JENNIFER L. BALES, MOUNTAIN VIEW PLAZA, 1520 EUCLID CIRCLE, LAFAYETTE, CO, 80026-1250, US

(21) Appl. No.: 11/073,047

(22) Filed: Mar. 4, 2005

Related U.S. Application Data
(63) Continuation-in-part of application No. 10/070,342, filed on Feb. 28, 2002, now abandoned.
(60) Provisional application No. 60/152,004, filed on Sep. 1, 1999.

(51) Int. Cl. A61B 18/14

(52) U.S. Cl. 606/39; 606/45

(57) ABSTRACT

An electro-surgical hand-held tool for cutting and cauterizing provides both impedance matching between the RF power source and the cutting tip, and field focusing, both within the hand-held device. The handheld unit includes a center-tapped coil wrapped around a core, and a capacitor. A switch signal is provided from the handheld unit to the RF power generator to signal the power generator to provide power.

This application is a continuation in part of U.S. application Ser. No. 10/070,342, filed Feb. 28, 2002, which claims benefit from provisional application for patent No. 60/152,004, filed Sep. 1, 1999 and PCT application No. PCT/US00/23874 filed Aug. 30, 2000.

### FIELD OF THE INVENTION

The present invention relates to apparatus and methods for cutting and cauterizing biological tissue. In particular, the present invention relates to a radio frequency electrosurgical device that cuts, cauterizes and ablates all in one unit with out the use of a grounding pad.

### DISCUSSION OF THE BACKGROUND ART

Many electrosurgical devices have developed over the years. One type of electrosurgical instrument includes a handheld cutting or cauterizing element having RF current applied to it. RF current experiences transmission line effects and losses, and hence the RF must be carefully impedance matched and field focused in order to get an efficient, narrow field.

U.S. Pat. No. 4,032,738 to Esty et al teaches a handheld electro-surgical instrument having an on/off switch on the handle, but no impedance matching or field focusing are provided within the hand held device. Therefore the efficiency and power of the device are low. U.S. Pat. No. 5,810,809 to Rydell teaches an arthroscopic shaver incorporating cauterization. The shaver portion uses a rotary motor and operates like a Dremel tool (suction removes the tissue). The cauterization is accomplished by applying a monopolar RF current to the tubular metal blade or a separate wire. Again, wires are run from an RF source to the cauterizing element, and no impedance matching or field focusing occur in the handheld device.

U.S. Pat. No. 5,807,392 to Eggers teaches a resistively heated cutting and coagulating tool. Some impedance matching is done in the handle of this device, via a transformer and a capacitor. No field focusing is required, since the device generates heat at tip 24 rather than a focused EM field.

FIG. 1 is a figure from U.S. Pat. No. 6,059,781 to Yamanashi et al, which teaches an electro-surgical device which cuts and cauterizes via a tip at which RF energy is focused. This device includes elements for impedance matching and field focusing. Impedance matching block 52 matches the impedance of the probe 51 with RF generator 44. Impedance matching device 52 is connected to RF power generator and to a Watts/Ampere meter 54. Meter 54 connects to loading and tuning coil 30. Coil 30 is connected to surgical instrument 51 via a heavily insulated cable 32, which is stated to be 110 cm long or a multiple of 22.

Impedance matching block 52 provides the majority of the impedance matching between the RF generator and the surgical instrument. The patent states in two places that it is desirable rotto have a coil in the operative field of the device, as this causes inconvenience to the surgeon. Yamanashi et al were not able to design impedance matching and/or field focusing circuitry that would fit within the handheld unit of the invention. Hence, they moved it entirely away from the handheld unit area. Unfortunately, both impedance matching and field focusing are dependent upon location and geometry. The field both attenuates and spreads over the distance from this circuitry to the cutting tip, reducing the effectiveness of the surgical device.

Therefore, if all of the impedance matching and field focusing could be accomplished by circuitry housed in a hand held unit, this would be a major advantage for surgeons and patients. First, fewer separate components and required connections between components results in easier setup and fewer things to go wrong. Second, every component in the equipment results in losses, driving up power requirements, and every component must be accounted for in impedance matching and transmission line effects. Third, locating the impedance matching and field focusing circuitry adjacent to the cutting tip means that the field can be tightly focused, resulting in a narrow cut, with more accuracy and quicker healing.

Until now, no one has accomplished good impedance matching and field focusing with circuitry housed within the handheld unit of an RF electrosurgical cutting and cauterizing device. Yamanashi et al U.S. Pat. No. 6,059,781, for example, requires a separate impedance matching unit 52, a Watts/Ampere meter 54, and a loading and tuning coil 30 in the cable leading to the handheld unit.

A need, therefore, remains in the art for methods and apparatus for an electro-surgical hand-held tool for cutting and cauterizing which provides both impedance matching between the RF power source and the cutting tip, and field focusing, both within the hand-held device.

### SUMMARY OF THE INVENTION

It is an object of the present invention to provide an electro-surgical hand-held tool for cutting and cauterizing which provides both impedance matching between the RF power source and the cutting tip, and field focusing, both within the hand-held device.

This object is accomplished by use of a single, center-tapped coil wrapped around a carefully designed core and a capacitor, all contained within the hand-held unit. The core provides a field focusing agent. The reduction in the amount of external circuit elements increases the efficiency of the unit and reduces its power requirements.

A specific benefit of the invention is that it provides a surgical device that can be used to cut, cauterize, coagulate, ablate and for vaporizing tissue and tissue reduction all in one unit. The device operates under water, as in saline conditions. It can be used to cut softer bone and cartilage, for example in shaving bone spurs.

### BRIEF DESCRIPTION OF THE DRAWINGS

FIG. 1 (Prior Art) is a block diagram showing a conventional electro-surgical device which cuts and cauterizes via RF energy.

FIG. 2 is a block diagram illustrating the electrical circuitry in an electro-surgical device according to the present invention.

FIG. 3 is a side cutaway view of the tip end of the electro-surgical device of FIG. 2.

FIG. 4 is a side cutaway view of the entire hand-held unit of the electro-surgical device of FIG. 2.

FIG. 5 is a side cutaway view of the cable connection end of the hand-held unit of the electro-surgical device of FIG. 2.

FIG. 6 is an isometric view of the connector of FIG. 5.

FIG. 7 is an exploded, transparent isometric view of the electro-surgical device of FIG. 2.

FIG. 8a is a partially sectioned side view of the coil connection element of FIG. 4.

FIG. 8b is a partially sectioned isometric view of the coil connection element.

FIG. 9 is a block diagram illustrating another preferred embodiment of the present invention, with a more complex switch in the handheld element.

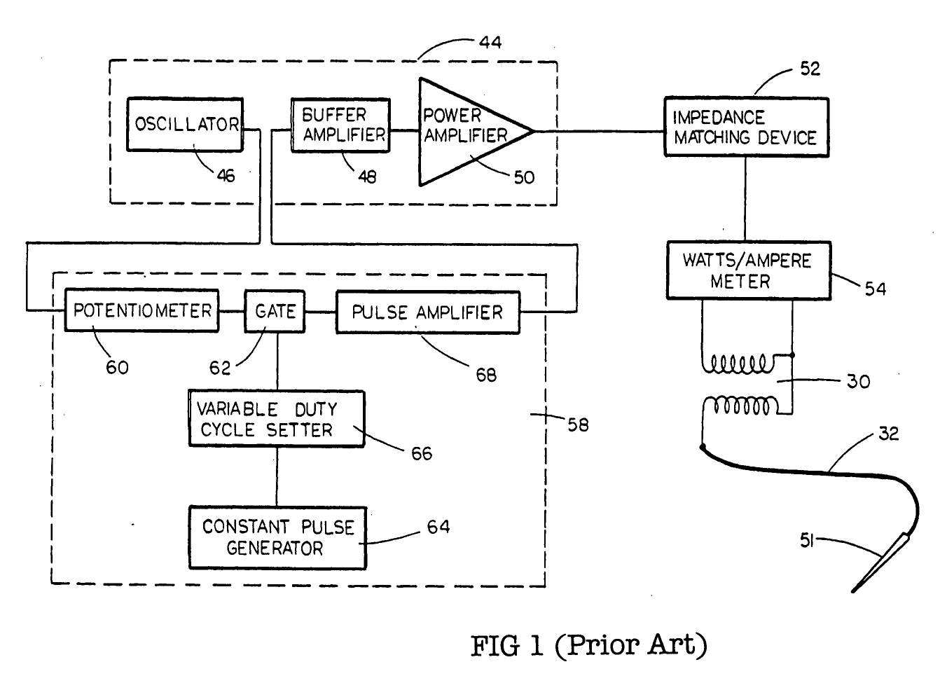

### DETAILED DESCRIPTION OF THE INVENTION

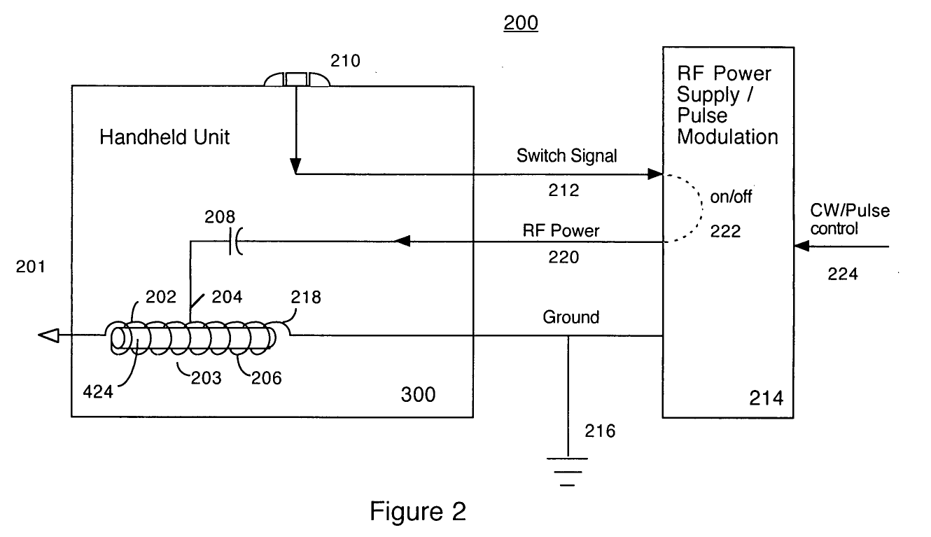

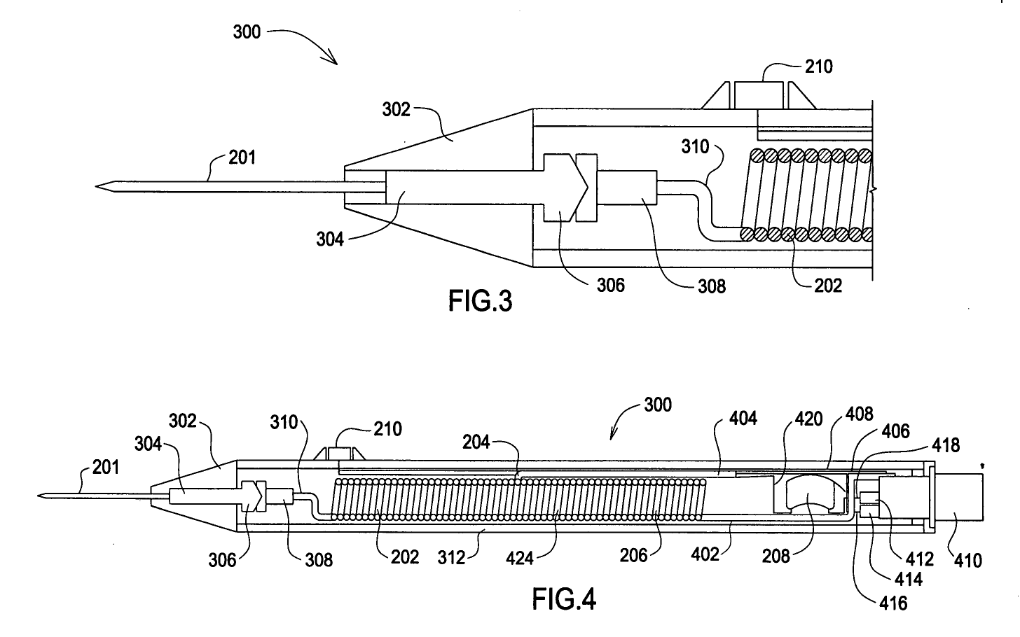

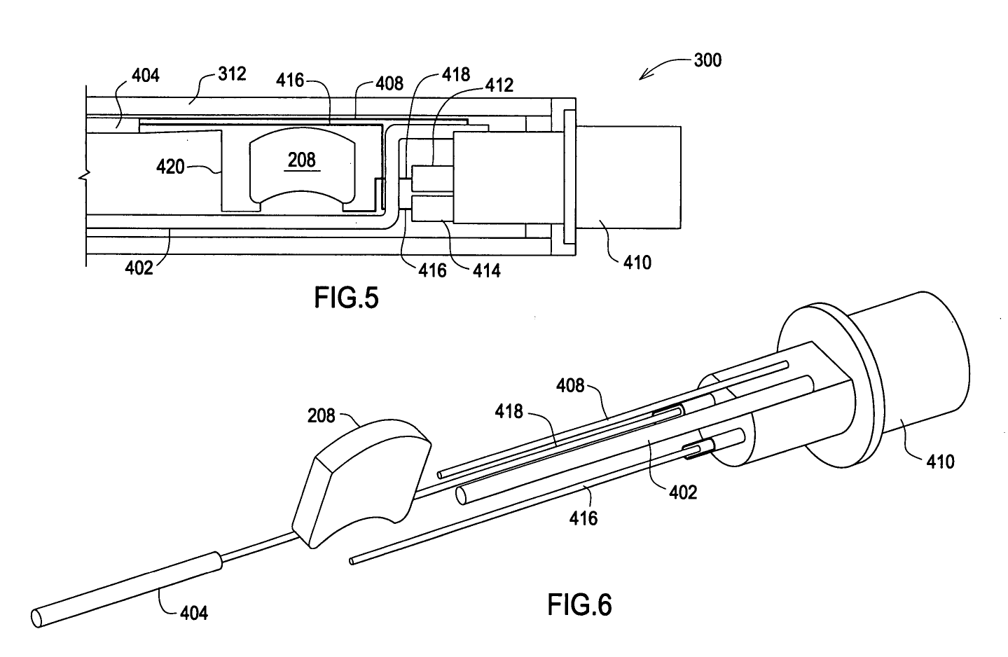

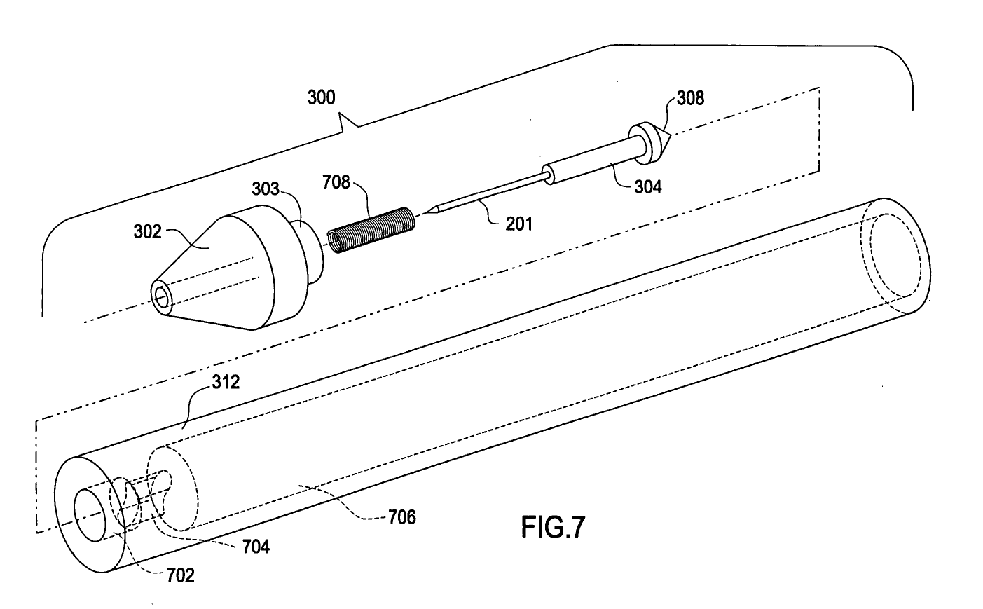

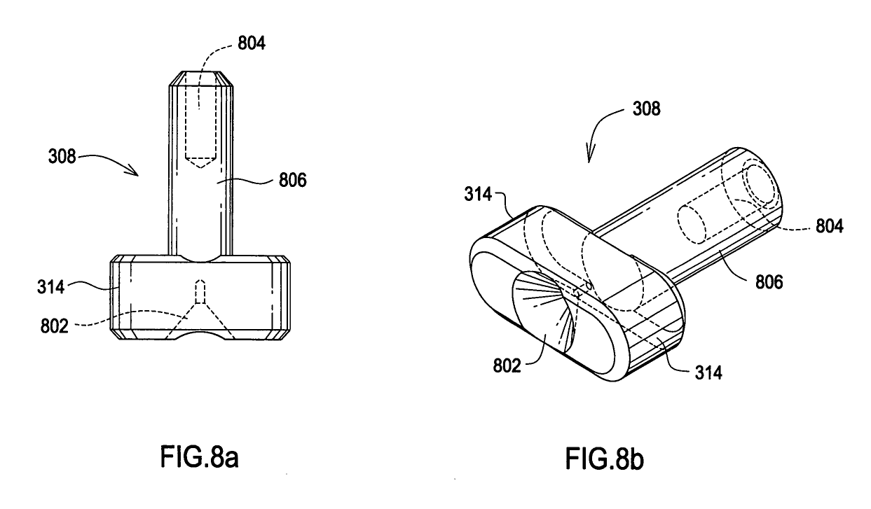

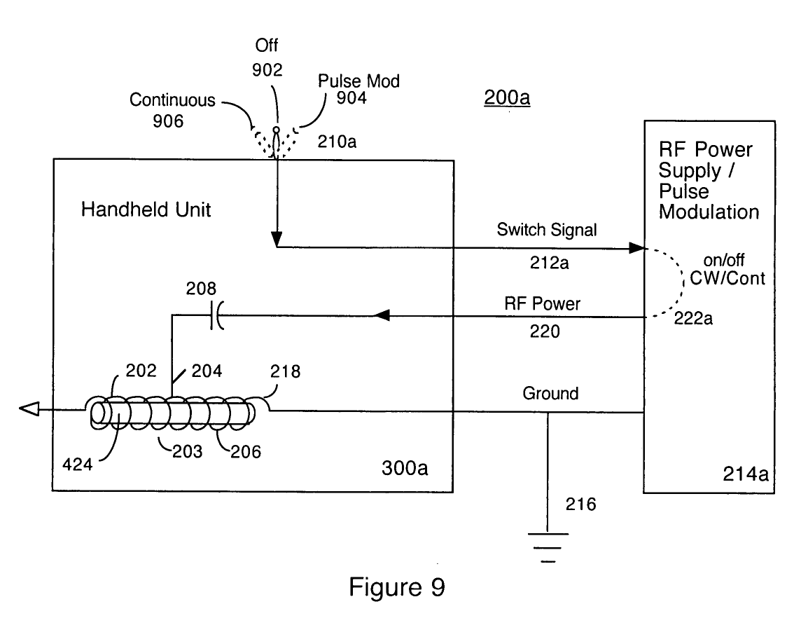

FIG. 2 shows the entire electrosurgical unit, including handheld element 300 and power supply 214. FIG. 3 shows the tip end of the handheld unit in detail. FIG. 4 shows the entire handheld unit, and FIG. 5 shows the connector end of the handheld unit in detail. FIG. 6 shows the connector in detail. FIG. 7 is an exploded view showing how the handheld unit goes together. FIGS. 8a and 8b show the coil connection element. FIG. 9 shows another embodiment of the electrosurgical device with a three position switch. Note that the terms “cutting,” “cutting device,” and “cutting tip” are used herein for convenience, but unless otherwise indicated are intended to include all the modes of the electrosurgical device: cutting, cauterizing, tissue reduction, ablation, etc.

FIG. 2 is a block diagram illustrating the electrical circuitry in electro-surgical device 200 according to the present invention. Tip 201 connects to the tip side 202 of coil 203. The ground side 218 of coil 203 connects to ground 216. The RF power for the device is provided by power source 214. In one preferred embodiment, power source 214 is a 13.56 Mhz RF power supply, for example the Advanced Energy RFX 600A power supply. This power supply operates on regular 60 Hz, 110V power, and generates output power up to 600 W. As the present invention requires under 100 W, this is ample. One surgery, on the soft palate of a dog, required only 30 W.

In another preferred embodiment, power source 214 comprises a 24 volt battery pack of the sort carried in a back pack, together with an intermediate power supply to step up the voltage and generate RF power. This portable embodiment is ideal for geographically remote applications, such as military forays. It is expected to weigh under 40 pounds for all of the equipment. The battery can be charged in a number of ways, including solar power, wind power, and the alternator of an engine. The battery will be able to operate the device for approximately two hours. The battery charges in about 30 to 45 minutes.

It is estimated that 10% of the soldiers killed on the battlefield bleed to death from extremity wounds. Many of these lives could be saved by the prompt and effective use of a tourniquet. However, a subset of extremity injuries occur at a level that is not amendable to tourniquet application. Vascular injuries in the region of the groin continue to be largely untreatable on the battlefield. Damage control surgery with the electrosurgical device of the present invention includes the ability to cut, cauterize, coagulate and ablate, and can save many of these lives by stopping excessive bleeding. In addition, use of a tourniquet usually results in loss of a limb, and should be avoided if a safe alternative exists. Again, quick, thorough cauterizing with electrosurgical device 200 can prevent the need for some amputations.

Switch 210 on the handheld unit generates switch signal 212 according to its position. When switch 210 is closed, signal 212 turns RF power supply on at 222, and RF power travels via RF power line 220 to center tap 204 of coil 203, via capacitor 208. In the embodiment of FIG. 2, control signal 224 controls whether power supply 222 generates a continuous wave signal (as is used in cutting and ablation) or a pulse modulated signal (as is used in cauterizing). Switch 210 has a common ground with coil 203 and power supply 214, as shown in FIG. 4.

Center tap 204 is located between the tip side 202 and the ground side 206 of coil 203. Preferably, the number of windings in each coil half 202, 205 is substantially equal. Coil 203 hence acts as an autotransformer. An autotransformer uses a single coil as the primary and secondary winding, with a center tap location determining the amount of step-up or step-down. In the case of coil 203, the configuration acts as a 2:1 step up transformer. A major advantage of this autotransformer configuration is the small space and the geometry it has. Coil 203 surrounds core 424, and both form a narrow cylinder which fits conveniently within handheld unit 300.

In use, a time-varying magnetic field induces a current which when impedance matched with a source will result in high current density at tip 201. Field focusing (accomplished primarily by core 424) results in a narrow, precise cutting field. The cutting field thus provides pinpoint heating of the tissue without undue spread of heat to surrounding tissue, resulting in better surgery and quicker healing. Necrosis in surrounding tissue is minimized with good field focusing.

Because convergence of energy is used for tissue heating, a grounding pad is not needed. The grounding (return) path is through hand-held unit 300 to ground 216 as shown.

While a specific cutting tip 201 is shown, different shaped and sized tips may also be used.

Ablation is the cutting off or removal of tissue. The rate of ablation is partly determined by “fluence”, that is the rate at which energy flows into the tissue. Using a high fluence level and short pulse width accomplishes ablation most effectively. Cauterizing uses heat to destroy a thin layer of tissue, preventing bleeding. Modulated pulses are used in cauterizing, for example in a 80:20 or 70:30 duty cycle.

In a preferred embodiment now described, each coil side 202, 206 comprises about 30 turns. From 20-40 turns for each coil side works well. If more turns are used, a finer gauge of wire is desirable, so that the circuitry continues to fit well within the handheld piece. Coil 203 is formed of, for example, 14 gauge coated copper wire. Core 424 is an iron powder core (not ferrite), e.g. carbonyl E, with a μᵣₑₗ of around 10, where B=μ₀μᵣₑₗH. B is magnetic flux density, μᵣₑₗ is the permeability of the material (μ₀ is the permeability of air), and H is magnetic field intensity, so the permeability of core 424 is directly related to field focus. The material permeability may vary from 9 to 20 in other embodiments.

In one embodiment, core 424 comprises two elements, each with outside diameter of ¼ inch and length of around 1 inch. Core elements this size are commercially available. A single core around 2 inches long could also be used. Core diameters of down to ⅛ inch work fairly well. Higher diameters, up to ½″ or more work well, but don't fit into the handheld device as well, and hence aren't as ergonomic. Similarly, longer cores, up to at least three inches total length, work well but aren't as ergonomic. Cores having total length of about 1.5 inches work fairly well. Core 424 also affects the Q of the device, which is a measure of the sharpness of the resonance peak.

The capacitance of capacitor 208 is about 50 Pico Farads. This can range from 25 PF to 100 PF, so long as the coil 203 is adjusted accordingly. Capacitor 208 is preferably a silver mica capacitor rated at 1 KV. With the properties of core 424, coil 203, and capacitor 208, together with inherent resistance, carefully selected and matched, very good results have been obtained without the need for variable capacitance or any impedance matching or field focusing outside of the elements shown, all of which are located within the hand-piece 300.

Note that the position of core 424 within coil 203 also affects the load impedance. Hence, a convenient way to adjust load impedance is to adjust the location of core 424. When the impedance is as desired, the position of core 424 is fixed in place. Of course a variable capacitor could be used to accomplish this, but the preferred embodiment uses a fixed silver mica capacitor rated at 1 KV. The voltage across capacitor 208 gets very high, and this type of capacitor stands up to the high voltages without arcing (punching through its dialectic barrier). Furthermore, variable capacitors can drift, whereas the silver mica capacitor of the preferred embodiment is very stable.

FIG. 3 is a side cutaway view of the tip end of the hand-held element 300 of electro-surgical device 200. The device includes a tip 201, a nose cone 302, and a housing 312. Cutting tip 201 is attached to tip connection element 304. Element 304 forms an electrical connection with coil connection element 308. In the embodiment of FIG. 3, element 304 includes a cone-shaped contact 306 which fits into a cone-shaped recess in element 308. This configuration has been shown to provide very good electrical contact.

Coil connection element 308 connects to the tip end 202 of coil 203. A switch 210 is wired as shown in FIGS. 4 and 5. In a preferred embodiment, switch 210 is a single pull single throw micro-switch, spring loaded so that when the surgeon releases the switch it is biased to turn itself off. Coil connection element 308 is preferably non-circular, so that when it is turned it is mechanically captivated within housing 312 (see FIGS. 7 and 8). Wire 310 is generally soldered to coil 203.

In a preferred embodiment, cutting tip 201 comprises tungsten and tip connection element 304 comprises bronze or phosphorus bronze. These materials have high current conductivity and won't melt or break down at high temperatures. Tip 201 is press fit into element 304. In this embodiment, coil connection element 308 is also bronze or phosphorus bronze, and forms a good electrical contact with element 304.

FIG. 4 is a side cutaway view of the entire hand-held unit 300 of electro-surgical device 200. The tip end elements have been described with respect to FIG. 3, and the connector end elements will be described with respect FIG. 5. Briefly, core 424 is disposed within coil 203. Core 424 is generally positioned approximately as shown, centered within coil 203. However, as discussed with respect to FIG. 2, the position of core 424 affects the load impedance, so it can be varied as required. RF ground wire 402 connects from the body of BNC connector 410 to the connector end 218 of coil 203. BNC connector 410 preferably connects via a 50 Ohm BNC triax cable to power source 214. Switch ground wire 406 leads from BNC 410 to switch 210. RF signal wire 418 connects to center tap 204 via capacitor 208. Switch signal wire 416 leads from BNC 410 to switch 210.

A preferred embodiment of the present invention comprises a handheld unit including housing 312, nose cone 302 and BNC 410, all having a length of no more than 9.5 inches, with an outside diameter of no more than ⅝ inch. Tip 201 extends out from the handheld element. All of the impedance matching circuitry and the field focusing circuitry (primarily core 424) are located within the housing 312.

FIG. 5 is a side cutaway view of the cable connection end of hand-held element 300. It shows the connection side elements in more detail. BNC Connector 410 includes two signal connections, connector 414 for the switch signal and connector 412 for the RF power input. The body of BNC connector 410 is grounded, so RF ground wire 402 and switch ground wire 408 connect to the BNC body. This is why no grounding pad is required. RF power travels via RF input wire 418, which connects to one side of capacitor 208, while the other end of capacitor 208 connects to wire 420, which in turn connects to copper wire 404, which is connected to center tap 204, e.g. via soldering. Switch signal wire 416 leads from switch 210 back to connector 410. When switch 210 is depressed, wire 416 carries the signal to RF power source 214 to provide RF power.

Note that RF power does not travel through switch 210, so that arcing is not a problem in this configuration.

FIG. 6 is a cutaway isometric view of connector 410 and its wiring within hand-held unit 300. In a preferred embodiment, wire 402 and wire 404 are insulated copper wire, e.g. 12 gauge, to form a good connection to coil 203. Switch wires 408, 416 are preferably 30 gauge copper or the like.

FIG. 7 is an exploded, partially sectioned isometric view of hand-held element 300. Housing 312 preferably includes an RF reflective material for shielding. This shielding prevents leakage of RF radiation (into or out of the device), and also improves field focus. One type of RF shielding which may be used is a thin foil-like sheeting material wrapped around the housing 312. Cavity 706 contains coil 203, core 424, a portion of BNC connector 410, and the wiring. BNC connector 410 caps the end of cavity 706, and is preferably press fit into cavity 706. Nose cone 302 has a threaded end 303 which screws into matching threads inside connect cavity 702.

Spring 708 biases contact 306 toward coil connection element 308 to maintain a good electrical contact between the two elements. Spring 708 may be formed of steel or plastic or the like. Contact 306 is disposed within connection cavity 702 as is the keyed portion 314 of coil connection element 308. Keyed portion 314 has an irregularly shaped cross-section (not circular) which matches key hole opening 704, so that once the keyed portion 314 is inserted through opening 704 and turned, it cannot retract through opening 704.

Note that the combination of spring 708 and keyed element 308 fix all the elements of handheld device 300 firmly in place. This is ideal in an RF device where any movement, or even compression of the coil, results in changes in the impedance of the device. This feature of the present invention precludes the need for on-the-fly adjustments made by the surgeon, such as were required in previous electrosurgical devices.

FIG. 8a is a partially sectioned side view of coil connection element 308. FIG. 8b is a partially sectioned isometric view of coil connection element 308. Coil connection element 308 has a non-circular cross section portion 314 to fit within keyed opening 704 (see FIG. 7), and a narrow cylindrical portion 806, which contacts coil wire 310. Cone shaped depression 802 mates with cone shaped contact 306. Coil wire 310 is inserted into wire solder point opening 804.

FIG. 9 is a block diagram illustrating another preferred embodiment 200a of the present invention, with a more complex switch 210a in the handheld element 300a. Switch 210a is a three position switch, for example a toggle switch. The three positions are off 902, continuous wave signal 906, and pulse modulated signal 904. Switch signal 212a now controls both whether the power supply 214a is on, and also whether it generates a CW or pulse signal.

1. An electrosurgical cutting device for cutting tissue, powered by an RF power supply, and comprising:
a hand held unit housing;
means for providing RF power from the power supply to the handheld unit housing;
means for providing a switch signal from the housing to the power supply;
a cutting tip emerging from the housing;
a switch on the housing for generating the switch signal; and
circuitry for impedance matching between the RF power supply and the tissue, and for providing field focusing of the RF power to the cutting tip, the circuitry including a capacitor, a coil having a center tap, and a core disposed within the coil, the circuitry disposed within the housing;
wherein the RF power is connected to the coil center tap through the capacitor, the cutting tip is connected to one end of the coil, and the other end of the coil is connected to a common ground; and
wherein the switch signal is designed to control the RF power flow.

2. The cutting device of claim 1 wherein the coil is an autotransformer on the order of a 2:1 step-up transformer.

3. The cutting device of claim 1 wherein the core is an iron powder core having a permeability of about 9-20.

4. The cutting device of claim 3 wherein core is formed of Carbonyl E.

5. The cutting device of claim 3 wherein core has an outside diameter of between around ⅛ inch to ½ inch, and a length of between about 1.5 inches to 3 inches.

6. The cutting device of claim 1 wherein the impedance of the device is adjusted by adjusting the position of the core within the coil.

7. The cutting device of claim 1 wherein the coil has a total of about 40-80 turns and is formed of coated copper wire.

8. The cutting device of claim 1 wherein the capacitor is a fixed capacitor having a voltage rating of at least on the order of 1000 volts.

9. The cutting device of claim 8 wherein the capacitor is about 25-100 Pico Farads.

10. The cutting device of claim 8 wherein the capacitor is silver mica capacitor.

11. The cutting device of claim 1 wherein the switch is a single pull single throw micro-switch.

12. The cutting device of claim 1, further comprising a coil connection element disposed between the tip and the coil, the coil connection element having a non-circular cross section portion, and wherein the housing forms a non circular opening for retaining the coil connection element.

13. The cutting device of claim 12, further comprising a nose cone attached to the housing from which the tip emerges and a spring between the tip and the nose cone for biasing the tip toward the coil connection element.

14. The cutting device of claim 1 wherein the tip comprises a cutting end portion of tungsten and a tip connection portion comprising one of either bronze or phosphorus bronze.

15. The cutting device of claim 1 wherein the switch further generates a signal designed to control whether the power supply generates continuous wave power or pulse modulated power.

16. An electrosurgical cutting device for cutting tissue comprising:
an RF power supply,
a hand held unit housing;
means for providing RF power from the power supply to the handheld unit housing;
means for providing a switch signal from the housing to the power supply;
a cutting tip emerging from the housing;
a switch on the housing for generating the switch signal; and
circuitry for impedance matching between the RF power supply and the tissue, and for providing field focusing of the RF power to the cutting tip, the circuitry including a capacitor, a coil having a center tap, and a core disposed within the coil, the circuitry disposed within the housing;
wherein the RF power is connected to the coil center tap through the capacitor, the cutting tip is connected to one end of the coil, and the other end of the coil is connected to a common ground; and wherein the switch signal controls the RF power flow.

17. The cutting device of claim 16 wherein the power supply is supplied by conventional AC power and provides at least 100 Watts of 13.56 Mhz power as the RF power.

18. The cutting device of claim 16 wherein the power supply comprises a battery and an intermediate power conversion device for stepping up the voltage from the battery and generating the RF power.

19. The cutting device of claim 16 wherein the means for providing RF power from the power supply to the handheld unit housing and the means for providing a switch signal from the housing to the power supply both comprise a BNC connector at the handheld device and a triax cable between the BNC connector and the power supply.

20. The cutting device of claim 19 wherein the triax cable is on the order of a 50 Ohm BNC cable.
